(19) United States
(12) Patent Application Publication
(10) Pub. No.: US 2010/0221294 A1
(43) Pub. Date: Sep. 2, 2010

(54) Use of Non-Straight Fiber Dispersed in a Composition and Compositions Thereof

(75) Inventors: John S. Kurek, Goshen, NY (US); Tao Zheng, Nanuet, NY (US); John R. Glynn, JR., Ridgewood, NJ (US); Harold E. Pahlick, Waldwick, NJ (US)

Correspondence Address: AVON PRODUCTS, INC., AVON PLACE, SUFFERN, NY, 10901, US

(73) Assignee: AVONE Products ,Inc., New York, NY (US)

(21) Appl. No.: 12/089,616

(22) PCT Filed: Dec. 14, 2006
(86) PCT No.: PCT/US2006/047764

§ 371 Date: Apr. 9, 2008

Related U.S. Application Data
(60) Provisional application No. 60/754,936, filed on Dec. 29, 2005.

(51) Int. Cl. A61K 8/02; A61K 8/00; A61Q 17/04; A61Q 5/12; A61Q 19/08; A61Q 1/02; A61Q 19/04; A61Q 1/10; A61K 8/72; A61K 8/73; A61K 8/81; A61K 8/88; A61K 8/84

(52) U.S. Cl. 424/401; 424/59; 424/63; 424/64; 424/70.7; 424/70.1; 424/70.13; 424/70.16; 424/70.17; 424/70.11

(57) ABSTRACT

Compositions and methods for enhancing, lengthening, volumizing, or concealment of keratinous surfaces or tissue. The compositions comprise non-straight fibers useful for formulating cosmetics, personal care products, and pharmaceutical compositions which are comfortable, durable, and overcome smudging, smearing, flaking, and detachment from keratinous surfaces, thereby resulting in an aesthetically pleasing appearance.

### FIELD OF THE INVENTION

The present invention relates generally to topically applied compositions which comprise non-straight fibers for enhancing the length, thickness, and volume of keratinous tissue, such as hair, and the concealment of fine lines or wrinkles on the skin surface. The non-straight fiber can be a natural or synthetic fibrous component, or a combination of both.

### BACKGROUND OF THE INVENTION

There is an increasing demand in the cosmetics industry to develop products that may be applied topically to keratinous surfaces or tissue, such as but not limited to, hair or skin, that improve the condition and aesthetic appearance of hair or skin. Consumers are also interested in mitigating or delaying the dermatological signs of chronologically-, hormonally-, or photo-aged skin, such as fine lines, wrinkles, drying, and sagging skin, however, especially wrinkling. In addition to covering or masking wrinkles, one may enhance or emphasize another feature, such as the eyes. Mascaras offer an alternative to directly hiding wrinkles by drawing attention to the eyes instead of fine lines or wrinkles on the face and neck.

By lengthening and thickening eyelashes, one may enhance the aesthetic appearance of the eyes. However, many mascaras which lengthen and thicken tend to smear, smudge, clump, and flake. These types of mascaras may also feel uncomfortable when applied. Thus, there remains a need to enhance features on the skin or hair, without the unwanted characteristics associated with current products.

The use of fibers in a cosmetic composition to lengthen, and thicken keratinous surfaces is known in the art. In particular, for example, mascaras with short, straight, and severely angled fibers have been used to provide long and thick eyelashes (U.S. Pat. No. 4,820,510; U.S. Patent Application Nos. 2002/0192251; 2004/0096473; JP Publication Nos. 57/158714; 03/153613; 07/179,323), including products such as MAYBELLINE XXL Volume+Length™ Microfiber Mascara (New York, N.Y.), TALIKA Lash Extender (Paris, France), and TWEEZERMAN® Fast Lash™ (Port Washington, N.Y.). Fibers in lipsticks, nail products, and other skin products are well known in the art for their properties in providing moisture, definition, nail repair, and smoothness (U.S. Pat. No. 5,498,407; EP-A-0 106 762; FR-A-1 529 329; JP-A-7/196440).

However, using these fibers in cosmetic compositions has presented various difficulties, such as clumping. Also, another disadvantage associated with these fibers is the difficulty in obtaining a volumizing effect in view of the sliding aspect on the surface of eyelashes, for example, of the fibers. Mascaras commonly used contain these short, straight, and/or severely angled fibers providing an undesirable appearance because they tend to smudge, smear, clump, flake and detach from eyelashes. Furthermore, such flaking may also make it difficult to apply more than one coat of the composition, as the application of subsequent coats may further act to detach fibers from the lashes.

Accordingly, there exists a need in the art to find a way to use fibers in a composition to obtain enhanced length, thickness, volume, or concealment for keratinous surfaces or tissues. It is also desirable to use fibers that possess characteristics for easy application of the composition forming a smooth uniform layer. In particular, there continues to be a need to formulate cosmetic compositions which achieve the desired aesthetic appearance of keratinous surfaces, such as a mascara that does not smudge, smear, clump, flake and detach from eyelashes, which thereby enhances and beautifies the eyelashes and the eyes. Furthermore, there is a need for a cosmetic composition that camouflages fine lines and wrinkles without excessive buildup, in order to provide an aesthetically appealing appearance.

### SUMMARY OF THE INVENTION

In accordance with the foregoing objectives and others detailed herein, the invention overcomes deficiencies associated with the prior art by providing in an embodiment of the invention, compositions comprising non-straight fibers in a physiologically acceptable medium. The non-straight fibers enhance keratinous surfaces by lengthening, thickening, volumizing, or concealing.

One embodiment of the invention comprises compositions with non-straight fibers including, for example, curved, sinusoidal, wavy, short waved, v-shaped, folded, crimped, crinkled, twisted, puckered, flagged, double-flagged, randomly-flagged, defined-flagged, undefined-flagged, split, double split, randomly split, multi-prong tipped, double multi-prong tipped, hooked, interlocking, cone shaped, symmetrical, asymmetrical, segmented, bi-material, multi-material, grooved, with appendages or arms, looped, shaped with different cross-section, or any combinations thereof.

In another embodiment, the composition is a cosmetic composition such as, but not limited to, a mascara, an eyebrow product, a lip product, a foundation, or a concealer.

A further embodiment relates to a method of enhancing, lengthening, and volumizing hair by applying the composition which has non-straight fibers and enabling the non-straight fibers to bind to hair and other fibers. This method thereby enhances the length and volume of hair upon application of the inventive composition to hair.

A further embodiment relates to a mascara which utilizes the inventive composition for enhancing the aesthetic appearance of the eyes by lengthening, thickening, and volumizing the eyelashes upon application of the mascara.

Yet another embodiment relates to a foundation or concealer which utilizes the inventive composition for concealing, camouflaging, or hiding fine lines or wrinkles on the skin upon application of the foundation or concealer.

These and other aspects of the present invention will become apparent to those skilled in the art after a reading of the following detailed description of the invention, including the illustrative embodiments and examples.

### BRIEF DESCRIPTION OF THE FIGURES

FIG. 1 shows non-limiting examples of non-straight fibers useful in the invention.

FIG. 2 shows non-limiting examples of cross sections of non-straight fibers useful in the invention.

### DETAILED DESCRIPTION OF THE INVENTION

The invention relates to a composition which comprises non-straight fibers in a physiologically acceptable medium for enhancing length, volume, thickness, or concealing, and the like by topically applying the composition to keratinous surfaces, such as but not limited to, hair and skin. Hair includes, but is not limited to, eyelashes, eyebrows, head hair, or any hair that is desired to be lengthened, thickened, and/or dyed.

A novel combination of elements produces a composition that is effective in lengthening, volumizing, and thickening, hair, such as for example eyelashes, and improving the aesthetic appearance of the eyelashes, or in concealing fine lines or wrinkles on skin. The composition of the invention comprises non-straight fibers dispersed in a physiologically acceptable medium or formulation. The composition may also comprise other ingredients, such as, but not limited to, an antioxidant, a preservative, an emulsifier, a solvent, a thickener, a hydrophobic/waxy, a colorant, or mixtures thereof.

“Straight fibers” as used herein includes fibers that are straight, cylindrical, symmetrical, and blunt-ended. “Straight fibers” as used herein also refer to fibers having an angle that is less than or equal to 15° and formed by folding the fiber at a point corresponding to half the length of the fiber.

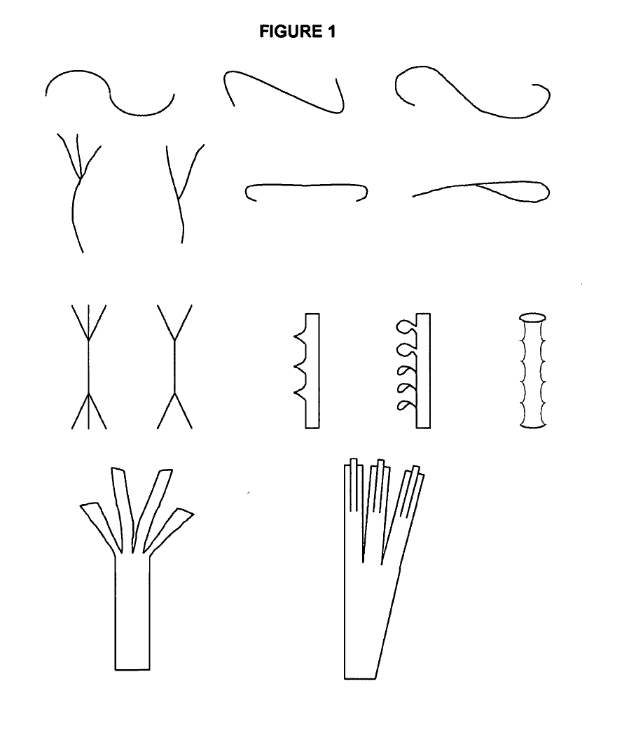

A “non-straight fiber” is not a “straight fiber” as used herein. “Non-straight” fibers may be short or long within the ranges provided herein, individual or organized, for example, hollow or solid. Hollow fibers may be preferred in order to achieve a lighter fiber with which to apply to the eyelashes, for example. Preferred non-straight fibers may include, but are not limited to, those with either one or both ends that are split, fingered, or pronged to enable the non-straight fiber to lengthen, thicken, volumize, conceal, and bind better to the keratinous surface and/or to other non-straight fibers. For example, “non-straight fibers” include but are not limited to, fibers that are curved, sinusoidal, wavy, short waved, U-shaped, V-shaped where the angle is greater than 15° but less than 180°, bent, folded, crimped, crinkled, twisted, puckered, flagged, double flagged, randomly flagged, defined flagged, undefined flagged, split, double split, multi-prong tipped, double multi-prong tipped, hooked, interlocking, cone shaped, symmetrical, asymmetrical, fingered, textured, spiraled, looped, leaf-like, petal-like, or thorn-like (see, FIG. 1).

Without being bound by theory, the non-straight fibers enhance volume by imparting a branched effect which gives the appearance of additional hairs. The non-straight fiber may have two or more extensions which when bound to an eyelash, for example, not only lengthen the eyelash but also volumize them by giving the appearance of more eyelashes. The interlocking or hooked fibers are particularly useful in lengthening eyelashes in that their unique shape allows multiple fibers to link together forming a chain. Thus, when an interlocking or hooked fiber, or part thereof, binds to an eyelash, the eyelash appears to be extended, as well as, thicker.

Another embodiment of the invention provides for a composition which utilizes the non-straight fibers to fill in fine lines, wrinkles, and grooves in the skin. Without wishing to be bound by theory, it is believed that the non-straight fibers fill the fine lines and wrinkles when the composition is applied to the skin. The non-straight fibers may more effectively carry or deliver pigments, thereby filling in the gaps and crevices in the skin, and essentially volumizing or “plumping” up the skin giving the appearance of more youthful skin. For example, a concealer or foundation comprising the unique non-straight fibers of the invention in addition to pigments, film formers, or other ingredients usually added to provide a smooth layer on the surface of skin. These fibers may deliver or carry pigments or other beneficial ingredients and fill in the fine lines and wrinkles giving the appearance of youthful skin.

These types of non-straight fibers enable the keratinous surface to which the fibers are bound to appear to have enhanced length and volume. A further advantage of using non-straight fibers is that only one application is necessary in order to produce the desired effects of enhanced length, thickness, volume, and concealment. By simply applying the inventive composition to the keratinous surface once, the user avoids the undesired effects of clumping and flaking, to name a few.

The non-straight fibers of the invention have a length that is not too short or very long because the fibers if too short or too long would not facilitate the lengthening or volumizing properties of the non-straight fibers. The non-straight fibers essentially have a length of about 0.1 millimeter to about 10 millimeters, preferably about 1 millimeter to 5 millimeters, more preferably from about 0.5 millimeter to about 3 millimeters, and most preferably from about 0.2 millimeter to about 2.5 millimeters. In yet another embodiment, the non-straight fiber is curvy or wavy, for example, a sinusoidal wave, where the length is 3π or any part thereof. The non-straight fibers of the composition may be present in a length including all values and subranges there between. In general, the non-straight fibers of the inventive composition have the same or substantially the same length. The non-straight fiber has a length that is greater than its diameter, where the diameter is the average of the cross-section of the non-straight fiber.

When the non-straight fiber is bent or crimped at a point that is half the length or at a point that is more or less than half the length of the fiber, the angle that is tangent to the longitudinal central axis is about 16° to about 179°, preferably about 20° to 120°, more preferably about 45° to about 90°. With respect to the wavy fibers which have a sinusoidal curve or part thereof, the amplitude may be greater than or less than about half the length of the fiber. The non-straight fibers of the composition may have an angle including all values and subranges there between.

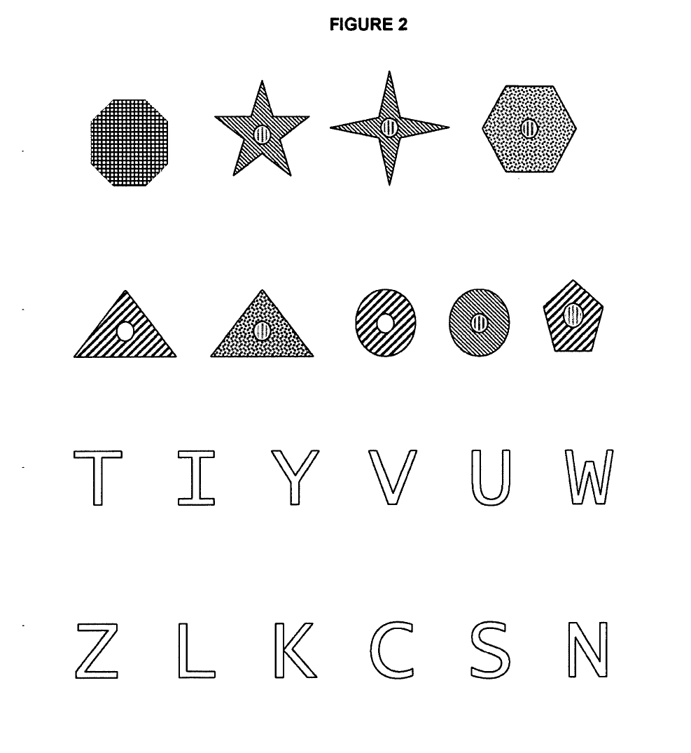

The non-straight fibers may have any shape and may especially have a circular or polygonal (square, hexagonal or octagonal) cross section depending on the intended specific application. Cross sections may be essentially any shape that is symmetrical or asymmetrical. Non-limiting examples of cross section shapes of non-straight fibers include a circle, a triangle, a square, a rectangle, a trapezoid, a parallelogram, a puzzle piece, a 3-, 4-, 5-, 6-petaled flower, a shape resembling essentially any letter of the Greek or Roman alphabet, or a shape having four or more sides. Preferably, the average diameter is averaged to be about 2 nanometers to about 500 micrometers, more preferably about 100 nanometers to about 100 micrometers, most preferably about 1 micrometer to about 50 micrometers. The non-straight fibers of the composition may have an average diameter including all values and subranges there between. Non-limiting examples of cross-sections of the non-straight fibers are shown in FIG. 2.

The weight or yarn count of the non-straight fibers is provided in denier or decitex and represents the weight in grams per 9 km of yarn. The non-straight fibers according to the invention preferably have a yarn count chosen in the range of from about 0.01 to about 10 denier, preferably from about 0.1 to about 2 denier, and more preferably from about 0.3 to about 0.7 denier. The non-straight fibers of the composition may have a weight including all values and subranges there between.

The non-straight fiber may be made of one type of material, two materials, or multiple materials. For example, the non-straight fiber may be segmented, grooved, with appendages or arms, shaped with different cross-section, or any combinations thereof. The segments may be divided into “pie wedge” or “citrus” pieces, where the different segments may alternate between two or more types of materials or may have all different materials. Another embodiment of the invention includes non-straight fibers having a multilayer structure comprising alternating layers of material, such as those described in documents EP 6 921 217, EP 686 858, and U.S. Pat. No. 5,472,798, incorporated herein by reference. Furthermore, the non-straight fibers may have a core that is a different material than the outer material. For example, the cross section of the fiber may be such that the inner core has a different material than the surrounding outer material. Moreover, the non-straight fiber may be hollow on the inside and have an outer material, where the cross section of the inner section does not necessarily reflect the shape of the cross section of the outer section of the non-straight fiber. Non-limiting examples of the combination of materials are shown in FIG. 2.

The non-straight fibers useful in the present invention may be selected from a variety of materials which are natural or synthetic, and where more than one material may be used in the non-straight fiber. Natural fibers include, but are not limited to, silk, cotton, wool, cork, sugar cane, flax, natural plant, vegetable, cellulose, modified cellulose fiber, and other keratin fibers. Non-limiting examples of synthetic fibers include polyester, rayon, nylon, polyamides, non-aromatic polyamides, aromatic polyimide-amides, acetate, rayon acetate, viscose, aramide, poly(p-phenylene terephtalamide), acrylic, acrylic polymer, such as but not limited to, poly(methyl methacrylate) or poly(2-hydroxyethyl methacrylate), polyolefin, such as but not limited to, polyethylene or polypropylene, glass, silica, carbon such as but not limited to, graphite, polytetrafluoroethylene, insoluble collagen, poly(vinyl chloride), poly(vinylidene chloride), poly(vinyl alcohol), polyacrylonitrile, chitosan, polyurethane, poly(ethylene phthalate), fiber optics, or blends thereof, such as, for example, polyamide/polyester blend.

In another embodiment, the non-straight fibers may be for example, resorbable synthetic fibers prepared from glycolic acid and caprolactone (Monocryl®; Johnson & Johnson; New Brunswick, N.J.); resorbable synthetic fibers of a copolymer of lactic acid and of glycolic acid (Vicryl®; Ethicon, Inc.; Somerville, N.J.); poly(terephthalic ester) (Ethibond Excel®; Ethicon, Inc.), deep grooved (Type 4DG™; Fiber Innovation Technology, Inc.; Johnson City, Tenn.), and stainless steel threads, blends thereof, and the like.

The surface of non-straight fibers may be pre-treated with a coating such that the non-straight fibers dispersed in a physiologically acceptable medium when applied onto keratinous surfaces provides an aesthetically appealing appearance. For example, the non-straight fibers may be coated with copper sulphide to induce anti-static effects, with polymers which enable the fibers to specifically arrange themselves, or with a coating that induces a color or a light-reflective property. The non-straight fibers may also be coated with a color or pigment to further enhance the aesthetic appearance of keratinous tissue, such as eyelashes or skin.

The non-straight fibers may, for example, be present in the composition in an amount ranging from about 0.01 weight % to about 30 weight %, preferably ranging from about 0.1 weight % to about 20 weight %, more preferably about 0.3 weight % to about 10 weight %, and most preferably, from about 0.5 weight % to about 3 weight %. The non-straight fibers or mixture thereof, and other ingredients, are uniformly distributed throughout the composition of the invention. The non-straight fibers of the composition may be present in an amount including all values and subranges there between.

A method of lengthening, thickening, and volumizing hair by applying the composition of the invention to hair; and enabling the non-straight fibers of the composition to bind to the hair, thereby lengthening, thickening, and volumizing hair when the composition is applied. For example, the composition is a mascara, which enhances the aesthetic appeal of the eyes by lengthening, thickening, and volumizing eyelashes without the undesired properties of smearing, smudging, flaking, or detachment of the mascara from the eyelashes.

A method of concealing fine lines or wrinkles on skin by applying the composition of the invention to skin; binding the non-straight fibers of the composition to the skin, thereby concealing or filling fine lines or wrinkles on the skin. For example, the composition is a foundation, concealer or lotion, which enhances the aesthetic appeal of the skin by concealing the fine lines or wrinkles on the skin without the undesired properties of smearing, smudging, flaking, or detachment of the skin product from the skin.

The compositions of the invention are comfortable, transfer resistant, flexible, and durable, which also lengthens, thickens, and volumizes hair, as well as, conceal or fill fine lines or wrinkles on skin. This technology and the inventive compositions are applicable to a wide variety products, including but not limited to: pigmented cosmetics, including foundations, concealers, mascaras, eye liners, eyeshadows, lipsticks, lip glosses, blushes; nail varnishes; hair colorants/dyes, hair sprays, gels and mousses, artificial tanning products, sunscreen lotions, moisturizing lotions, lotions with active ingredients, and fragrance. The products of the present invention are particularly useful in any cosmetic, personal care, or pharmaceutical application to keratinous surfaces.

In particular, the composition of the invention may include a cosmetic formulation. One embodiment of the invention relates to cosmetic foundations, where the formulation of a cosmetic foundation may contain, in addition to the composition of the invention, additional thickening agents and emollients in an amount that provides coverage and achieves the other desired properties for camouflaging fine lines and wrinkles of the skin. This coverage is a result of the non-straight fibers, which fill the fine lines and wrinkles, thereby giving the appearance that the skin is full, wrinkle-free, and youthful.

Another embodiment of the invention is a mascara, which employs the composition of the invention and produces increased stability and adherence to keratin surfaces, such as eyelashes. These mascaras provide greater wear resistance, attachment, and improved cosmetic properties which enhance the length, volume, and thickness of eyelashes. These properties result in a more desirable aesthetic appearance which is greatly desired in mascaras.

Some of the desirable features of mascaras in particular include, but are not limited to, the ability to thicken and lengthen eyelashes while still allowing the eyelashes to look natural, while resisting smudging and flaking. In addition to being smudge-proof, it is desirable for mascara to be transfer resistant (i.e., resist unwanted removal while remaining easy to remove when desired). Optionally, the mascara may be water-resistant. Moreover, as the mascara is applied to the eyelashes in close proximity to the sensitive eye and certainly in the sensitive eye area, the mascara should not cause allergic reactions. An additional benefit would be for the mascara to promote healthy eyelashes by making them softer and smoother, and promoting their growth. To achieve this, typically natural conditioners, such as for example, lanolin or bisabolol or synthetic conditioners, like glycols or vitamin E, are added to the mascara. Overall, the ideal mascara should beautify the eye by enhancing the appearance of the eyelashes around the eye.

Non-straight fibers may be used in any mascara formulation. Non-limiting examples of different types of mascaras include water soluble, waterproof, two-step, three-step, cream, gel, weightless, colorless, tinted, or glittered mascaras. For example, two-step mascaras comprise a base coat and a top coat, where the non-straight fibers are in either the base or top coat. Either coat may be pigmented or non-pigmented depending on the particular embodiment. Furthermore, the non-straight fibers may or may not be colored.

In one embodiment, the two-step mascara utilizes a base coat having the non-straight fibers and colorless as the first coating on eyelashes. Then a top coat which is preferably pigmented with properties which may include, but is not limited to, water insoluble, glossy, and weightless, is applied onto the first coating. The two-step mascara comprising non-straight fibers enhances the length and volume of eyelashes.

Another embodiment utilizes a three-step mascara process. The three-step process involves first priming the eyelashes with a light coat of mascara; adding the non-straight fibers to the eyelashes that are slightly wet from the first coat, allowing the non-straight fibers to bind to the eyelashes; and applying a second coat of mascara or a top coat to the eyelashes. The three-step method is fairly easy to apply for a natural or more dramatic look.

A further embodiment of the invention includes lotions such as moisturizers, suntan lotion, sunblock, or artificial suntans or tints. These lotions employing the composition of the invention provide smooth and even coverage of the lotion. The non-straight fibers assist in this coverage by filling in the fine lines and wrinkles of the skin. Lotions using the composition of the invention also provide greater wearability.

Yet another embodiment of the invention includes eyeliner products. Eyeliners employing the composition of the invention may provide increased adherence and even application to eyelid tissue. Eyeliners using the composition of the invention may also provide improved cosmetic properties.

Another embodiment is a make-up composition for the lips employing the composition of the invention. These lip products provide a homogeneous film that has a light texture and remains comfortable to wear throughout the day. The preferred lip make-up is not tacky or sticky, nor does it transfer, migration, or stain, but is long-lasting, soft, supple, elastic, flexible, and comfortable on the skin. The non-straight fibers of the lip product fills the fine lines in lips and especially those in advanced aging lips, thereby providing smooth and even coverage.

In a one embodiment, the compositions may provide a barrier between the skin and the environment, where the barrier contains the active and/or functional ingredients. The film formed by the composition may increase the activity of the functional ingredients such as the SPF and UV light protection and/or block the effect of the humidity and the environment.

In one embodiment of the invention, additional ingredients may be added to the composition thereby providing a comfortable composition when applied to the keratinous surface, as well as, to hold or bind onto the surface, topical coatings, actives and functional ingredients. The active or functional ingredients may include colorants, pigments, ultraviolet filters, moisturizing agents, fragrance, insecticides, pharmaceutical agents and other active or functional ingredients known in the cosmetic or pharmaceutical arts.

For example, the composition of the invention may additionally include any additive usually employed in the field envisaged such as antioxidants, perfumes, essential oils, stabilizers, emulsifiers, cosmetic active substances, moisturizers, vitamins, essential fatty acids, lipophilic sunscreens, liposoluble polymers, and especially hydrocarbon polymers such as polyalkylenes and polyacrylates for improving smoothness or spreadability, or other cosmetic or pharmaceutical properties desired by one of skill in the art. Non-limiting examples of optionally added ingredients include: emollients, thickening agents, for example, clays, or organoclays, silicas, cellulose derivatives, plasticizers, gels, oils, waxes, preservatives, solvents, surfactants; hectorites; synthetic polymers such as an acrylic polymer or an associative polymer of the polyurethane type; gums and in particular xanthan gum; spreading agents; dispersants; preservatives, in particular water-soluble preservatives; antifoaming agents; wetting agents; ultraviolet-screening agents; perfumes; fillers; cosmetic or pharmaceutical active agents; moisturizers; vitamins and derivatives thereof; and biological materials and derivatives thereof. If the softness and elasticity of the corn position are to be increased still further, it is also possible to add a plasticizer which is commonly added for cosmetic materials. Suitable materials may include both low-molecular weight and also high-molecular weight plasticizers which are optionally used, solubilized, or dissolved in a co-solvent.

Suspending and thickening agents typically include waxes, silica gels, gums, clays, fumed silica, fatty acid soaps, and various hydrocarbon gels, and other ingredients that when incorporated into the formulation remain on the surface of keratinous tissues. Non-limiting examples of ingredients, such as emollients, that may preferably be used in the compositions of the invention include glycerine, propylene glycol, cyclomethicone, dimethicone, and emollients and other similar ingredients disclosed in the International Cosmetic Dictionary and Handbook Vols. 1 and 2. Eds. Wenninger, J. A. and G. N. McEwen, Cosmetic, Toiletry, and Fragrance Association, Washington D.C., 2000, which is hereby incorporated by reference.

For colored or pigmented products, the ratio of thermoplastic film former, tackifier resins, volatile solvents, and additional ingredients may be adjusted for maximizing adherence to and water, oil, and transfer resistance of the keratinous substrate or surface. An important consideration is the ratio of pigments to the amount of film former. A pigment should be understood to mean inorganic or organic, white or colored particles. Coloring agents that may be used in the practice of the invention may include pigments, lakes, and dyes which are well known in the art and are disclosed in the Cosmetic Ingredient Handbook, First Edition, J. M. Nikitakis, et al., Cosmetic, Toiletry, and Fragrance Association, Washington D.C., 1988, the contents or which are hereby incorporated by reference.

Non-limiting examples of organic pigments include, FD&C dyes, D&C dyes, including D&C Red, Nos. 2, 5, 6, 7, 10, 11, 12, 13, 30 and 34, D&C Yellow No. 5, Blue No. 1, Violet No. 2. Exemplary inorganic pigments include, but are not limited to, metal oxides and metal hydroxides such as magnesium oxide, magnesium hydroxide, calcium oxide, calcium hydroxides, aluminum oxide, aluminum hydroxide, iron oxides (α-Fe2O3, y-Fe2O3, Fe3O4, FeO), red iron oxide, yellow iron oxide, black iron oxide, iron hydroxides, titanium dioxide, titanium lower oxides, zirconium oxides, chromium oxides, chromium hydroxides, manganese oxides, cobalt oxides, cerium oxides, nickel oxides and zinc oxides and composite oxides and composite hydroxides such as iron titanate, cobalt titanate and cobalt aluminate. Other suitable coloring agents include ultramarine blue (i.e., sodium aluminum silicate containing sulfur), Prussian blue, manganese violet, bismuth oxychloride, talc, mica, sericite, magnesium carbonate, calcium carbonate, magnesium silicate, aluminum magnesium silicate, silica, titanated mica, iron oxide titanated mica, bismuth oxychloride, and any other pigment or treated pigment known in the cosmetic arts.

Fillers and mother-of-pearl may also be added to the inventive compositions to modify the texture of the composition and the matte/gloss appearance. Fillers should be understood to mean lamellar or nonlamellar, inorganic or synthetic, colorless or white particles. Mother-of pearl should be understood to mean iridescent particles produced especially by certain mollusks in their shell or else synthesized. Pearling agents that may be used in the practice of the invention include mica, iron oxides, titanium dioxide and any other pearling agent known in the cosmetic arts.

Although some of these materials may include an oily feeling and increased spreadability, as observed with some esters and organic sunscreens, the overall composition of the invention maintains its desired properties of lengthening, thickening, volumizing, durability, applicability, wearability, uniformity, sheen or gloss, drying time, preferably in the absence of irritation. The person skilled in the art will of course take care to choose the optional additional compounds and/or their quantities in such a way that the advantageous properties of the composition according to the invention are not, or are substantially not, impaired by the envisaged addition(s). In embodiments where these materials are added to the compositions of the invention to enhance the spreadability and the emollience of the product, however, it is preferred that the above materials be present in low enough concentrations for the composition to retain its desired properties. These ingredients may be selected variously by the person skilled in the art in order to prepare a composition which has the desired properties, for example, consistency or texture. The choice of additional ingredients and their concentrations may also be adjusted to vary the desired properties.

A further embodiment of the invention relates to a composition, preferably a topical liquid, gel, foam, cream, or lotion that is a cosmetic, a pharmaceutical or medicinal formulation, an insect repellent, or a sun product, where the composition comprises non-straight fibers. The inventive composition is comfortable, transfer resistant, flexible, and durable, which lengthens, thickens, and volumizes, as well as, conceals or fills fine lines or wrinkles, and is capable of binding and/or delivering one or more active components, such as but not limited to, a colorant, a dye, a ultraviolet absorber, a moisturizer, a biologically active agent, a insecticide/pesticide, and an organic or inorganic active agent. For example, the composition of the present invention may be resistant, including treated swimming pool water, fresh water, and ocean water. The composition is also smudge resistant and does not flake. The composition may be used in products, such as but not limited to, sun care, skin care, color cosmetics, mascaras, hair products (shampoos, conditioners, hairspray, mousses and dyes/colorants), a nail enamel, a lip coloring product, a lip gloss, a foundation, eye make-up, a skin care product, a personal hygiene product, and a topical drug or active delivery.

The packaging and application device for any embodiment of the invention is chosen and manufactured by persons skilled in the art on the basis of their general knowledge, and adapted according to the nature of the composition to be packaged. Moreover, the type of device to be used may be in particular linked to the consistency of the composition, in particular to its viscosity; it may also depend on the nature of the constituents present in the composition, such as the presence of volatile compounds.

In yet a further embodiment, a method of conveying enhanced length, thickness, volume, and the like by applying the composition of the instant invention having non-straight fibers, to keratinous tissue such as but not limited to hair and skin, in an amount effective to enhance the length, thickness, and volume of hair, or conceal fine lines or wrinkles.

It will be evident to those skilled in the art that the invention can take many forms, and that such forms are within the scope of the invention as claimed. The spirit and scope of the appended claims therefore should not be limited to the description of the preferred versions contained herein.

The inventive composition may be used in a variety of cosmetic formulations comprising an effective amount of non-straight fibers, necessary to obtain the desired properties. The skilled artisan will be able to determine the effective amount depending on the application and degree of durability, applicability, wearability, uniformity, adhesion, transfer resistance, and abrasion resistance, preferably in the absence of irritation. One skilled in the art will also be able to determine the amount and type of non-straight fibers, and additional ingredients needed to obtain a stable cosmetic product, depending on the application. A stable cosmetic product is one of sufficient stability to enable effective commercialization of the cosmetic product.

### EXAMPLES

The following examples further describe and demonstrate embodiments within the scope of the present invention. The examples are given solely for the purpose of illustration and are not to be constructed as limitations of the present invention, as many variations thereof are possible without departing from the spirit and scope of the invention.

### Example 1

### Mascara Formulation

TABLE 1

| INGREDIENTS | % |
|---|---|
| WATER | q.s. 100% |
| POLYMER | 10.00 |
| PRESERVATIVE | 1.00 |
| PIGMENT | 14.00 |
| EMOLLIENT | 9.00 |
| WAX | 12.50 |
| NON-STRAIGHT FIBERS | 2.00 |

Any composition comprising ingredients normally used in the formation of mascaras and the addition of non-straight fibers may be used. For example, the mascara composition has water, a preservative, polymer or copolymer, solvent, tackifier resins, film formers, waxes, moisturizers, conditioners, pigments, and the non-straight fibers of the invention. In a water insoluble mascara, the mascara composition has alcohol, a preservative, polymer or copolymer, solvent, tackifier resins, film formers, waxes, moisturizers, conditioners, pigments, and the non-straight fibers of the invention.

### Example 2

### Foundation Formulation

Any corn position comprising ingredients normally used in formulating foundations with the addition of non-straight fibers may be used.

TABLE 2

| INGREDIENTS | % |
|---|---|
| WATER | q.s. 100% |
| THICKENER | 1.00 |
| PRESERVATIVE | 0.65 |
| BUTYLENE GLYCOL | 13.00 |
| PIGMENT | 15.00 |
| SUNSCREEN | 2.00 |
| EMULSIFIER | 0.70 |
| POLYMER | 15.00 |
| NON-STRAIGHT FIBERs | 1.00 |

### Example 3

### Measurement of Lengthened Hair

A mascara containing non-straight fibers, according to Example 1 of the present invention is prepared, and a mascara without the non-straight fibers is prepared. Both mascaras are tested using a panel of 10-20 female individuals. Qualifying panelists are selected over the age of 18. They have eyelashes medium in length, e.g., about 0.6 to about 0.9 cm. The chosen panelists participate in a two day double blinded study and evaluations are carried out before application of the mascara for a baseline and immediately after application of the mascara.

The panelists wear no makeup or moisturizer on the day of testing. Each panelist applies 4-14 strokes of the mascara to their upper lashes with one brush having either of the two prepared mascaras and 4-14 strokes to their lower lashes with a second brush having either of the two prepared mascaras. The mascara is applied onto the brush without pumping the brush in the mascara container. Images of the eyelashes are obtained using a digital camera at a ratio of 1:2.5. An image of each the right and left eye are recorded. The baseline image is used as a guideline for placement for the following visits to ensure reproducibility. The images are digitized and analyzed to determine the “perceived eyelash length”. An average of the length of five of the same eyelashes at each time point is made to determine the “perceived eyelash length.” The results of the analysis demonstrate that the keratinous surface is enhanced by an increase over the baseline for the length of the lashes.

### Example 4

### Measurement of Wrinkles

A cosmetic formulation containing non-straight fibers, according to Example 2 of the present invention is prepared, and a foundation without the non-straight fibers is prepared. Both cosmetic formulations are tested using a panel of 10-20 female individuals. Qualifying panelists are selected over the age of 18. The chosen panelists participate in a double blinded study and evaluations are carried out before application of the cosmetic formulation for a baseline, immediately after application of the cosmetic formulation. The panelists wear no makeup or moisturizer on the day of testing.

The panelists are evaluated visually, by wrinkle count, and wrinkle length/depth.

Visual Evaluation by four (4) evaluators who make gross visual comparisons of the appearance of wrinkling as seen on the left (treated) side vs. the appearance of wrinkling on the right (untreated) side of the face. The Evaluators use a score of 0 to 4 with “No change” being ‘0’ to “difference visible at a distance” being ‘4’.

Wrinkle count is based on analysis of “before” and “after” digital photographs of the treated side of the face (orbital area).

Wrinkle length based on analysis of “before” and “after” digital photographs of the treated side of the face (orbital area) is measured. Wrinkle depth is measured by taking a silicone replica/mask of the face “before” and “after” treatment.

Each panelist applies a thin layer of the cosmetic formulation of Example 2 to the face. An image of each the right and left orbital area and face are recorded before and after treatment. The baseline image is used as a guideline for placement for the following visits to ensure reproducibility. The images are digitized and analyzed to determine the “perceived wrinkle length”. An average of five wrinkles is measured is made to determine the “perceived wrinkle length.” The results of the analysis demonstrate that the keratinous surface is enhanced by an increase over the baseline for the length of the wrinkles.

A further method for measuring the number of wrinkles, wrinkle length, and depth of the wrinkles, is by facial topography. These evaluation factors may be also measured by making an impression of the face using a silicone polymer mask before and after treatment. This method is preferably performed after digital imaging. A silicone replica is made of the patients for four minutes in which time the silicone polymer sets. After about 5 minutes, the hardened silicon replica is removed from the panelist's face. The skin surface replica provides a baseline negative impression (a mold) and record of the skin surface to which the silicone polymer set.

The baseline silicone replica is compared to the subsequent replica obtained from the panelist after treatment. The silicone replica is placed on a horizontal surface on a table under a digital imaging camera. The replica is illuminated by light from a light source orientated at an angle (35° is a preferred angle) from the horizontal (and perpendicular to the major skin lines) thereby generating shadows due to the negative impressions of lines, wrinkles and furrows in the skin present on the replica surface. The light is incident upon the negative skin surface replica at the angle. The digital camera is connected to a computer equipped with, for example, Quantirides software (version 2.0, Monaderm, Monaco). The Quantirides software can generate and analyze the imaged skin surface topography impression, as shown by the silicon replica. The following parameters can be calculated by the software: mean depth (micrometer), mean length (mm), total length (mm), number of wrinkles, surface area of wrinkles (depth×length; mm2) and form factor (ratio length/depth) and used to obtain results.

Thus, this example demonstrates that the facial topography method set forth in this example can be used to determine the effects of the inventive composition comprising non-straight fibers.

All evaluators may see clearly visible improvement in most of the panelists. The wrinkle count of completely disappeared may result in a decrease in the total number of whole wrinkles counted. The wrinkle length is a measurement of wrinkles that are diminished but not gone. Panelists may have a decreased wrinkle length of those wrinkles that are not diminished. The wrinkle depth measures the wrinkles embedded in the mask before and after treatment. The wrinkle depth is reduced such that there are barely any indications of wrinkles.

The content of all patents, patent applications, published articles, abstracts, books, reference manuals and abstracts, as cited herein are hereby incorporated by reference in their entireties to more fully describe the state of the art to which the invention pertains.

It should be understood that the foregoing description is only illustrative of the present invention. Various alternatives and modifications can be devised by those skilled in the art without departing from the invention. Accordingly, the present invention is intended to embrace all such alternatives, modifications and variations that fall within the scope of the appended claims.

What is claimed is:

1. A composition comprising non-straight fibers in a physiologically acceptable medium, wherein the non-straight fibers thereby enhance keratinous surfaces by lengthening, thickening, volumizing, or concealing.

2. The composition according to claim 1, wherein the non-straight fibers comprise: a curved, a sinusoidal, a crimped, a bent, a V-shaped, a short wave, a flagged, a split, a fingered, a multi-prong tipped, a U-shaped, a hooked, an interlocking, or cone-shaped fiber.

3. The composition according to claim 2, wherein the non-straight fibers comprise: a double flagged, a randomly flagged, an undefined flagged, a double split, or a double multi-prong tipped fiber.

4. The composition according to claim 2, wherein the non-straight fibers are flagged, split, fingered, or pronged on one or both ends of the non-straight fibers.

5. The composition according to claim 2, wherein the sinusoidal fiber has a length of 3π, or fragment thereof.

6. The composition according to claim 1, wherein the non-straight fibers have a length ranging from about 0.1 millimeter to about 10 millimeters.

7. The composition according to claim 1, wherein the non-straight fibers have a yarn count from about 0.01 to about 10 denier.

8. The composition according to claim 1, wherein the non-straight fibers have a cross section shape comprising a circle, a triangle, a square, a rectangle, a trapezoid, a parallelogram, a puzzle piece, a 3-, 4-, 5-, 6-petaled flower, a shape resembling essentially any letter of the Greek or Roman alphabet, or a shape having four or more sides.

9. The composition according to claim 8, wherein the cross section has an average diameter ranging from about 2 nanometers to about 500 micrometers.

10. The composition according to claim 1, wherein the non-straight fibers are treated and/or coated.

11. The composition according claim 1, wherein the non-straight fibers comprise silk fiber, cotton fiber, wool fiber, cork fiber, sugar cane fiber, flax fiber, natural plant fiber, vegetable fiber, cellulose fiber, modified cellulose fiber, keratin fiber, polyester fiber, rayon fiber, nylon fiber, polyamide fiber, non-aromatic polyamide fiber, aromatic polyimide-amide fiber, acetate fiber, rayon acetate fiber, viscose fiber, aramide fiber, poly(p-phenylene terephtalamide) fiber, acrylic fiber, acrylic polymer fiber, poly(methyl methacrylate) fiber, poly(2-hydroxyethyl methacrylate) fiber, polyolefin fiber, polyethylene fiber, polypropylene fiber, glass fiber, silica fiber, carbon fiber, graphite fiber, polytetrafluoroethylene fiber, insoluble collagen fiber, poly(vinyl chloride) fiber, poly(vinylidene chloride) fiber, poly(vinyl alcohol) fiber, polyacrylonitrile fiber, chitosan fiber, polyurethane fiber, poly(ethylene phthalate) fiber, fiber optics fiber, resorbable synthetic fiber prepared from glycolic acid and caprolactone, resorbable synthetic fiber of a copolymer of lactic acid and of glycolic acid, poly(terephthalic ester) fiber, deep grooved fiber, stainless steel fiber, or blends thereof.

12. The composition according to claim 1, wherein the non-straight fibers are present in the composition in an amount ranging from about 0.01 weight % to about 15 weight %.

13. The composition according to claim 1, wherein the composition when applied to hair lengthens, thickens, and volumizes the hair.

14. The composition according to claim 1, wherein the composition when applied to skin conceals fine lines or wrinkles of the skin.

15. The composition of claim 1, wherein the composition is a cosmetic composition comprising a mascara, an eyebrow product, a hair product, a lip product, a skin product, a lotion, a foundation, a concealer, or an artificial suntan product.

16. The composition of claim 1, further comprising an ingredient comprising a colorant, a sunscreen, a moisturizer, a biologically active agent, or an organic or inorganic active agent.

17. A mascara comprising non-straight fibers in a physiologically acceptable medium, wherein the non-straight fibers thereby enhance keratinous surfaces by lengthening, thickening, volumizing, or concealing, and wherein the non-straight fibers comprise: a curved, a sinusoidal, a crimped, a bent, a V-shaped, a short wave, a flagged, a split, a fingered, a multi-prong tipped, a U-shaped, a hooked, an interlocking, or cone-shaped fiber.

18. A method of lengthening, thickening, and volumizing hair, comprising:
a) applying the composition of claim 1 to hair; and
b) binding the non-straight fibers of the composition to hair,
thereby lengthening, thickening, and volumizing hair when the composition is applied.

19. The method of claim 18, wherein hair are eyelashes, eyebrows, or head hair.

20. The method of claim 18, wherein the composition is a mascara, eyebrow product, or hair product.

21. A method of concealing fine lines or wrinkles on skin, comprising:
a) applying the composition of claim 1 to skin; and
b) binding the non-straight fibers of the composition to the skin, thereby concealing fine lines or wrinkles on skin.

22. The method of claim 21, wherein the composition is a lip product, a skin product, lotion, foundation, a concealer, or artificial suntan product.

23. A method of lengthening, thickening, and volumizing eyelashes, comprising:
a) applying one coat of a mascara to eyelashes;
b) applying the composition of claim 1 to the mascara coated eyelashes; and
c) applying a second coat of the mascara to the eyelashes,
thereby lengthening, thickening, and volumizing the eyelashes.
